(19) United States
(12) Patent Application Publication
(10) Pub. No.: US 2026/0337142 A1
(43) Pub. Date: Oct. 29, 2026

(54) PROCESS FOR PRODUCING ACRYLIC ACID FROM HEAVY WASTE BYPRODUCTS

(71) Applicants: Dow Global Technologies LLC, Midland, MI (US); Rohm and Haas Company, Collegeville, PA (US)

(72) Inventors: Vipul Sharma, King of Prussia, PA (US); Kirk W. Limbach, Dresher, PA (US); Thomas Goodall, Houston, TX (US); Daniel J. Martenak, Perkasie, PA (US); Reetam Chakrabarti, Phoenixville, PA (US); Joseph F. Dewilde, King of Prussia, PA (US)

(21) Appl. No.: 19/391,336

(22) Filed: Nov. 17, 2025

Related U.S. Application Data
(60) Provisional application No. 63/728,466, filed on Dec. 5, 2024.

(51) Int. Cl. C07C 57/04; B01J 19/00; C07C 51/347; C07C 51/42; C07C 57/13

(52) U.S. Cl. CPC C07C 57/04; B01J 19/006; C07C 51/347; C07C 51/42; C07C 57/13

(57) ABSTRACT

A process for recovering acrylic acid from Michael addition products comprises feeding a liquid stream to a vapor-liquid separation vessel, wherein the liquid stream comprises acrylic acid and Michael addition products of acrylic acid. A vapor stream comprising acrylic acid is removed from the vapor-liquid separation vessel, and a bottoms stream comprises the Michael addition products of acrylic acid. The bottom stream comprising the Michael addition products of acrylic acid is fed to a dimer cracker reactor. Acrylic acid is recovered from a vapor stream exiting the dimer cracker reactor.

### FIELD OF THE INVENTION

The invention relates to a method for preparing acrylic acid from heavy waste byproducts.

### BACKGROUND OF THE INVENTION

Acrylic acid and its ester derivatives are used in the manufacture of a broad spectrum of polymer products found across multiple market segments. For example, poly-acrylic acid (PAA) formulations are commonly found in detergent applications, sodium salts of PAA are used as super absorbent polymers (SAP) found in baby diapers and other adult hygiene products. Copolymers of acrylic monomers with methacrylic monomers, styrene, butadiene and other functionalized vinyl monomers are used to manufacture dispersions and resins that are ubiquitous in consumer and industrial products such as architectural, industrial, paper and automotive coatings, performance additives for concrete admixtures and asphalts, textiles, adhesives, sealants and caulks to name just a few applications.

Commercially, acrylic monomer production is dominated by acrylic acid which serves as a platform chemical for a broad range of acrylate derivatives. Acrylic acid may be produced using a variety of different methods. For example, acrylic acid can be made via propylene oxidation, bio-based processes (e.g., using any one of glycerol, 3-hydroxypropionic acid, lactate, lactic acid, etc., as a starting material), the ethylene cyanohydrin process, the acetylene process (i.e., the Reppe process), the β-propiolactone process, acrylonitrile hydrolysis, or coupling carbon dioxide with ethylene or ethane. Industrially, most of the global capacity of acrylic acid is manufactured via the two-stage heterogeneously catalyzed oxidation of propylene over mixed metal oxide catalysts.

The crude acrylic acid (CAA) that is produced by any of the known methods contains Michael addition products, which include dimers, trimers, and higher-mers of acrylic acid. These Michael addition products may reversibly react to form acrylic acid or higher or lower oligomers.

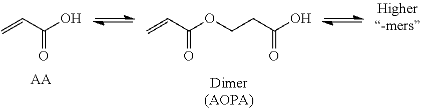

Michael addition products of acrylic acids are also present in waste streams and bottoms streams from acrylic acid purification processes, as well as waste and bottoms streams from ester production processes that use acrylic acid in the formation of the esters, such as, for example, butyl acrylate production.

Dimer crackers are typically used to recover acrylic acid value from the Michael addition products present in these streams.

Attempts have been made to improve acrylic acid recovery in dimer crackers.

U.S. Patent Application Publication No. 2004/0220427 discloses a process in which a dimer cracker operates at a high temperature and short residence time.

U.S. Pat. No. 7,569,721 discloses a process for conducting equilibrium-limited reactions using two reaction zones. The first reaction zone retains a least a portion of the product in a liquid phase, and at least a portion of the liquid from the first reaction zone is introduced into a second reaction zone to crack heavies and to vaporize at least a portion of the product.

U.S. Pat. No. 6,252,110 discloses a process for recovering acrylic acid from high boiling impurities by introducing the high boiling impurities into an acrylic acid recovery column, distilling acrylic acid from the column top and introducing bottom liquids from the acrylic acid recovery column into a pyrolyzing tank.

U.S. Pat. No. 6,482,981 discloses the use of a crystallization step to melt crystallize the acrylic acid and separate the acrylic acid into purified acrylic acid and a residual mother liquid, which is fed to an acrylic acid dimer decomposition step.

There is a need in the art for improved recovery of acrylic acid from Michael addition products.

### SUMMARY OF THE INVENTION

The invention is directed to a process for recovering acrylic acid from Michael addition products comprising:
a) feeding a liquid stream to a vapor-liquid separation vessel, wherein the liquid stream comprises acrylic acid and Michael addition products of acrylic acid;
b) removing a vapor stream comprising acrylic acid from the vapor-liquid separation vessel and a bottoms stream comprising the Michael addition products of acrylic acid;
c) feeding the bottom stream comprising the Michael addition products of acrylic acid to a dimer cracker reactor; and
d) recovering acrylic acid from a vapor stream exiting the dimer cracker reactor.

### BRIEF DESCRIPTION OF THE DRAWINGS

FIG. 1 is a schematic diagram showing the process according to an embodiment of the present invention.

FIG. 2 is a graph of the viscosity of the bottoms stream as a function of the percentage of trimers and higher-mers in the bottoms stream.

### DETAILED DESCRIPTION OF THE INVENTION

All percentage compositions are weight percentages (wt %), and all temperatures are in °C, unless otherwise indicated. Averages are arithmetic averages unless otherwise indicated. An “average concentration” is the arithmetic average of the concentration entering a region and the concentration exiting the region, where the region is an individual reactor, a reactor system, or a zone within a reactor or reactor system. An “average ratio” is the ratio of the average concentration of one component relative to the average concentration of another component.

As used herein, the terms “Michael addition products of acrylic acid” and “dimer, trimer, and higher-mers of acrylic acid” refer to oligomers of acrylic acid. The oligomers of acrylic acid can reversibly react to form higher or lower oligomers, as well as acrylic acid.

The present invention relates to a process for recovering acrylic acid from heavy waste byproducts, such as Michael addition products of acrylic acid.

In the inventive process, acrylic acid recovery in the dimer cracker is improved by driving the reaction towards the formation of acrylic acid by removing acrylic acid from a liquid stream before the liquid stream enters the dimer cracker. By removing the acrylic acid, the reaction will drive the formation of acrylic acid from 3-(acryoloyloxy)propanoic acid (AOPA) to acrylic acid to a greater degree than would have occurred, thus increasing the recovery of AOPA as acrylic acid. This will in turn drive the reaction of the trimer to AOPA, as well as the cracking of any higher-mers present in the dimer cracker to lower oligomers.

The liquid stream fed to the distillation column comprises acrylic acid and Michael addition products of acrylic acid. Preferably, AOPA is the majority component of the Michael addition products. More preferably, AOPA is the majority component in the liquid stream comprising acrylic acid and its Michael addition products.

According to the present invention, a liquid stream comprising acrylic acid and Michael addition products are fed to a vapor-liquid separation vessel to remove acrylic acid from the liquid stream. A vapor stream comprising acrylic acid is removed from the vapor-liquid separation vessel. A bottoms stream comprising the Michael addition products is removed from the bottom of the vapor-liquid separation vessel.

Water may be removed from the liquid stream before entering the vapor-liquid separation vessel. Alternatively, the vapor-liquid separation vessel may be used to remove the water with the vapor stream. Preferably, the liquid stream fed to the vapor-liquid separation vessel comprises less than 5 wt % of water based on the total weight of the liquid stream. More preferably, the liquid stream comprises less than 3 wt %, even more preferably less than 2 wt %, yet more preferably less than 1 wt %, and still more preferably less than 0.5 wt % of water based on the total weight of the liquid stream.

Preferably, the vapor-liquid separation vessel comprises a plurality of baffles to aid the separation of the vapor phase containing the acrylic acid and the liquid phase containing the Michael addition products of acrylic acid.

The liquid stream fed to the vapor-liquid separation vessel may be sprayed into the vapor-liquid separation vessel.

The vapor phase exiting the vapor-liquid separation vessel may be condensed in a condenser to form a liquid overhead product stream comprising acrylic acid. Preferably, at least a portion of the overhead product stream is recycled to the vapor-liquid separation vessel as reflux. Preferably the reflux ratio is at least 0.1, more preferably at least 0.5.

The vapor-liquid separation vessel is preferably maintained at a temperature of at least 150 °C. More preferably, the vapor-liquid separation vessel is maintained at a temperature of at least 165 °C.

The bottoms stream comprises 5 to 95 wt % of AOPA and other acrylic acid Michael addition products, including trimers and tetramers of acrylic acid based on the total weight of the bottoms stream. Preferably, at least 50 wt % of the Michal addition products of acrylic acid in the bottoms stream comprise AOPA. Preferably, the bottoms stream comprises 20 to 95 wt % of AOPA and other acrylic acid Michael addition products, more preferable 40 to 95 wt % of AOPA and other acrylic acid Michael addition products, and even more preferably 60 to 95 wt % of AOPA and other acrylic acid Michael addition products based on the total weight of the bottoms stream.

A polymerization inhibitor may be added to the vapor-liquid separation vessel along with the liquid stream. Alternatively, a polymerization inhibitor may be added to the process prior to the vapor-liquid separation vessel. The polymerization inhibitor may comprise any known polymerization inhibitors to prevent unwanted polymerization of the acrylic acid. Examples of preferred inhibitors include soluble manganese ions, soluble copper ions, hydroquinone (HQ), monomethyl ether hydroquinone (MEHQ), phenothiazine (PTZ), 2,2,6,6-tetramethyl-1-piperidinyloxy (TEMPO) and related compounds such as 4-hydroxy TEMPO. Combinations of inhibitors can be employed, such as, for example, a mixture of a source of soluble manganese ions, such as manganese acetate, and 4-hydroxy TEMPO. Molecular oxygen or air may also be used, as oxygen is known to be an inhibitor. The inhibitor is employed in an amount sufficient to prevent or reduce the polymerization of acrylic acid, as is well known to those skilled in the art.

The bottoms stream comprising the Michael addition products of acrylic acid are fed to a dimer cracker reactor, where the Michael addition products are cracked to form acrylic acid and lower oligomer Michael addition products. For example, tetramer can be cracked to form trimer, and trimer can be cracked to form AOPA, while AOPA is cracked to form acrylic acid. Because a large percentage of the acrylic acid is removed in the vapor stream exiting the vapor-liquid separation vessel, the reversible reaction of acrylic acid and Michael addition products favors the production of acrylic acid and lower oligomers of Michael addition products in the dimer cracker compared to a dimer cracker in which the percentage by weight of acrylic acid entering the dimer cracker reactor is higher.

The dimer cracker reactor may comprise a single vessel or more than one vessel. For example, the dimer cracker reactor may comprise a continuously stirred tank reactor, or a continuously stirred tank reactor followed by a plug flow reactor. The vapor-liquid separation vessel may be part of the dimer cracker reactor. For example, the vapor-liquid separation vessel may comprise a pipe-like structure at the top of the dimer cracker reactor. Alternatively, the vapor-liquid separation vessel may be separate from the dimer cracker reactor.

The dimer cracker reactor may be catalyzed or uncatalyzed. Preferably, the dimer cracker reactor is uncatalyzed.

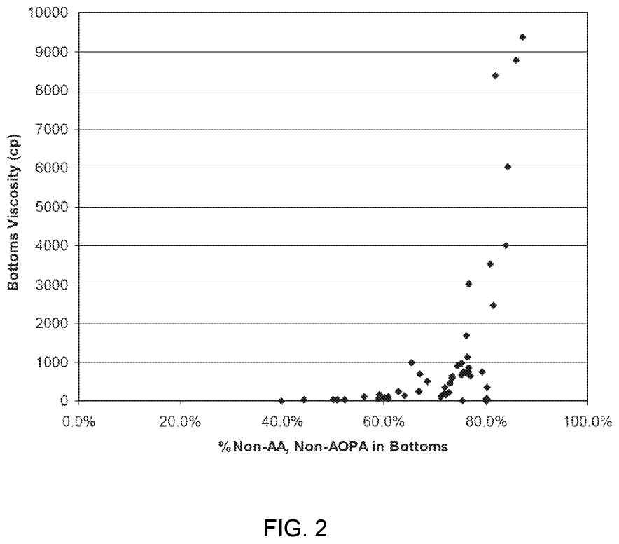

As shown in FIG. 2, the viscosity of the bottoms stream can vary greatly based on the amount of non-acrylic acid and non-AOPA in the bottoms stream. The viscosity of the bottoms stream increases significantly when the amount of trimer and tetramer Michael addition products increases.

By driving the reversible reaction to favor formation of acrylic acid and lower oligomers, it may be easier to control the viscosity of the dimer cracker reactor. As trimer is reduced to acrylic acid and AOPA, the viscosity of the reactor mixture may be maintained at an operable level.

In the vapor-liquid separation vessel, the viscosity of the bottoms stream can be controlled by adjusting the pressure of the vapor-liquid separation vessel. Alternatively, the viscosity may also be adjusted with temperature or by adding a lower-viscosity material, such as an oil.

Preferably, the dimer cracker reactor is maintained at a temperature of at least 150 °C, more preferably at least 165 °C.

Acrylic acid is recovered in the vapor stream of the dimer cracker reactor. Preferably, the recovered acrylic acid is returned to the vapor-liquid separation vessel.

The residence time in the vapor-liquid separation vessel and dimer cracker reactor is preferably less than 2 hour, more preferably less than 0.5 hour.

Preferably, the liquid stream used in the present invention comprises acrylic acid generated by any known acrylic acid production process. For example, acrylic acid can be made via propylene oxidation, a bio-based process (e.g., using any one of glycerol, 3-hydroxypropionic acid, lactate, lactic acid, etc., as a starting material), an ethylene cyanohydrin process, an acetylene process (i.e., the Reppe process), a β-propiolactone process, an acrylonitrile hydrolysis process, or a process performed by coupling carbon dioxide with ethylene or ethane. The reaction mixture produced during the acrylic acid production process may be processed in one or more unit operations. These operations may include, for example, cooling processes, quenching processes, separations, purifications, etc., or a downstream reaction such as an ester production process.

Preferably, the liquid stream used in the inventive process comprises a low grade acrylic acid stream, a waste stream, or a bottoms stream from a purification process that draws off a product stream that is substantially comprised of acrylic acid, such as, for example, a product stream comprising at least 90 wt % acrylic acid. The liquid stream may also, or alternatively, comprise a waste stream or bottoms stream from an ester production process, preferably an alkyl acrylate production process. The low grade acrylic acid stream, waste stream, or bottoms stream comprises Micheal addition products of acrylic acid and at least some amount of acrylic acid from the acrylic acid production process.

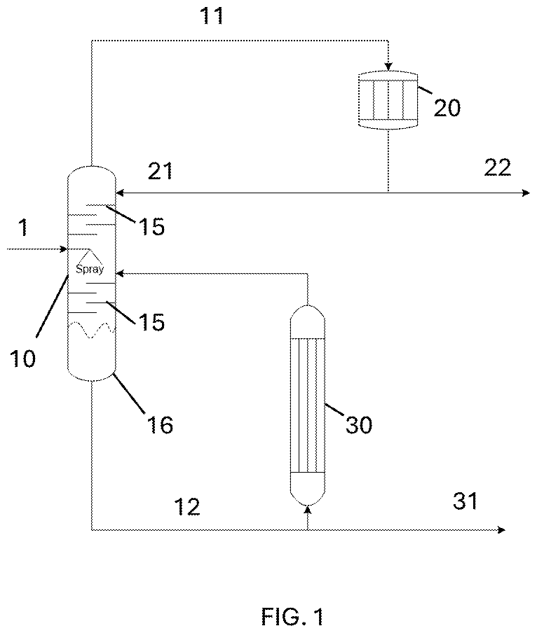

One embodiment of the present invention is shown in FIG. 1. A liquid stream 1 comprising acrylic acid and Michael addition products of acrylic acid is fed into vapor-liquid separation vessel 10 comprising a plurality of baffles 15. Vapor stream 11 is condensed in condenser 20. A portion of the condensed vapor stream 21 is returned to vapor-liquid separation vessel 10 as reflux, and the remainder is drawn off as product stream 22. The bottoms collect in sump 16 and bottoms stream 12 containing the Michael addition products exits the bottom of the vapor-liquid separation vessel 10. Bottoms stream 12 is fed to dimer cracker reactor 30 to convert AOPA to acrylic acid and higher oligomers to acrylic acid and AOPA. The vapor stream 31 exiting dimer cracker 30 is returned to vapor-liquid separation vessel 10.

1. A process for recovering acrylic acid from Michael addition products comprising:
a) feeding a liquid stream to a vapor-liquid separation vessel, wherein the liquid stream comprises acrylic acid and Michael addition products of acrylic acid;
b) removing a vapor stream comprising acrylic acid from the vapor-liquid separation vessel and a bottoms stream comprising the Michael addition products of acrylic acid;
c) feeding the bottom stream comprising the Michael addition products of acrylic acid to a dimer cracker reactor; and
d) recovering acrylic acid from a vapor stream exiting the dimer cracker reactor.

2. The process of claim 1, wherein the bottoms stream comprising the Michael addition products of acrylic acid comprises 5 to 95 wt % 3-(acryoloyloxy)propanoic acid and Michal addition products of acrylic acid based on the total weight of the bottoms stream.

3. The process of claim 1, wherein the vapor-liquid separation vessel comprises a plurality of baffles.

4. The process of claim 3, wherein the liquid feed is sprayed into the vapor-liquid separation vessel.

5. The process of claim 1, further comprising controlling the viscosity of the bottoms stream by adjusting the pressure of the vapor-liquid separation vessel.

6. The process of claim 1, wherein a polymerization inhibitor is fed to the vapor-liquid separation vessel.

7. The process of claim 1, wherein the vapor-liquid separation vessel and the dimer cracker are maintained at a temperature greater than 150 °C.

8. The process of claim 7, wherein the vapor-liquid separation vessel and the dimer cracker are maintained at a temperature greater than 165 °C.

9. The process of claim 1, wherein condensed reflux from the vapor stream of the vapor-liquid separation vessel is recycled to the vapor-liquid separation vessel at a reflux ratio of at least 0.1.

10. The process of claim 9, wherein condensed reflux from the vapor stream of the vapor-liquid separation vessel is recycled to the vapor-liquid separation vessel at a reflux ratio of at least 0.5.

11. The process of claim 1, wherein the dimer cracker comprises a continuously stirred tank reactor.

12. The process of claim 1, wherein the liquid stream comprises 5 wt % water based on the total amount of the liquid stream entering the vapor-liquid separation vessel.

13. The process of claim 1, wherein acrylic acid in the liquid stream comprising acrylic acid is produced by a method selected from the group consisting of propylene oxidation-based process, a bio-based process, an ethylene cyanohydrin-based process, an acetylene-based process, a β-propiolactone-based process, an acrylonitrile hydrolysis-based process, or a process performed by coupling carbon dioxide with ethylene or ethane.
